(19) United States
(12) Patent Application Publication
(10) Pub. No.: US 2004/0132643 A1
(43) Pub. Date: Jul. 8, 2004

(54) Histone deacelylase inhibitors in diagnosis and treatment of thyroid neoplasms

(76) Inventors: Antonio Tito Fojo, Rockville, MD (US); Susan Elaine Bates, Bethesda, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(21) Appl. No.: 10/250,320

(22) PCT Filed: Jan. 9, 2002
(86) PCT No.: PCT/US02/00714

(51) Int. Cl. A61K 38/12; A61K 31/19

(52) U.S. Cl. 514/10; 514/575

(57) ABSTRACT

Disclosed herein are novel approaches to thyroid cancer therapy. These approaches include methods to enhance thyroid specific gene expression, for example methods to enhance expression of thyroglobulin and/or the Na⁺/I⁻ symporter in thyroid cancer cells. Enhanced expression of thyroid-specific genes promotes cellular differentiation and reduces biologically aggressive behavior such as invasion and metastasis. In addition, enhanced expression of thyroglobulin and/or the Na⁺/I³¹ symporter increases the ability of thyroid cancer cells to concentrate iodine or iodide, thereby making the cells more susceptible to radioactive iodine therapy. Also disclosed herein are methods for detecting thyroid neoplasms in a subject, by administering a therapeutically effective amount of a histone deacetylase inhibitor, administering a detectable agent whose uptake or concentration in thyroid cells is increased by administration of the histone deacetylase inhibitor, and detecting the detectable agent.

### FIELD

This disclosure relates to the field of diagnosis and treatment of thyroid neoplasms, specifically to the use of histone deacetylase inhibitors in the diagnosis and treatment of thyroid neoplasms.

### BACKGROUND

The thyroid gland is located in the neck of mammalian subjects, and is divided into two lateral lobes connected by a small central isthmus. The lobes are divided by fibrous septa into pseudolobes composed of spherical structures called follicles, which consist of a single layer of epithelial cells (follicular cells) surrounding a lumen (see FIG. 1). The follicular lumen is filled with a colloid material consisting of over 75% thyroglobulin, the precursor protein molecule for thyroid hormones. See Larsen et al., The Thyroid Gland, Chapter 11 in Williams' Textbook of Endocrinology, J. Wilson editor, 1998.

The thyroid's follicular cells produce two active thyroid hormones, triiodothyronine (T3) and thyroxine (T4). Structurally, the thyroid hormones are coupled tyrosine residues modified to contain three or four iodine atoms. They are formed via a multistep process in the thyroid follicular cells. The follicular cells express thyroglobulin (TG) polypeptide, take up and concentrate iodide anions, and iodinate tyrosyl residues within the TG polypeptide chain. Tyrosyl iodination in TG yields monoiodotyrosine (MIT; one iodine atom) and diiodotyrosine (DIT; two iodine atoms). MIT and DIT residues are then coupled in a process termed the coupling reaction. T3 and T4 released into the blood after proteolytic cleavage from TG.

Thyroid hormone biosynthesis requires that the thyroid actively takes up and concentrates iodine. To accomplish this task, thyroid follicular cells express the sodium iodide symporter (Na⁺/I⁻ symporter or NIS), a membrane protein that cotransports sodium and iodide into the thyroid follicular cell. NIS concentrates iodine in follicular cells about 100-fold over levels found in plasma. TG assists in the concentrating process, by serving as a repository or sink for organified iodine in the cell.

A hypothalamic-pituitary-thyroid feedback loop regulates thyroid hormone production and secretion. The hypothalamus generates thyroid-releasing hormone (TRH) to stimulate synthesis and release of pituitary thyroid-stimulating hormone (TSH). TSH stimulates production and release of thyroid hormones into the blood. In turn, thyroid hormones provide negative feedback control by reducing TRH and TSH release.

TSH stimulates growth of follicular cells and regulates gene expression in follicular cells. TSH up regulates the expression of thyroid specific genes, including thyroglobulin, NIS, and thyroid peroxidase (TPO). Dai et al., Nature 379: 458-461, 1996; Suzuki et al., Proceedings the National Academy Of Sciences USA 95: 8251-8256, 1998; Ulianich et al., J. Biol. Chem. 274: 25099-25107, 1999; De La Vieja et al., Physiological Reviews 80: 1083-1105, 2000. TSH's enhancement of thyroid-specific gene expression is one way in which TSH stimulates increased production of thyroid hormones.

Current Therapeutic Approaches to Thyroid Cancer

Thyroid cancer is the most common endocrine malignancy and accounts for the majority of deaths from endocrine cancers. In the United States alone, approximately 17,200 new cases of thyroid cancer are diagnosed each year, and about 1500 deaths are attributable to the disease.

Currently, conventional thyroid cancer therapy includes surgical resection (thyroidectomy) to remove the primary tumor. This is generally followed by radioactive iodine (¹³¹I) treatment, which exploits the thyroid cell's ability to concentrate iodine. These measures are followed by continuous therapy with oral thyroid hormone supplementation, to suppress any remaining thyroid by reducing pituitary production of TSH. Conventional therapy may be supplemented by other measures, such as external beam irradiation and chemotherapy. See Schlumberger et al., New England Journal of Medicine 338: 297-3006, 1998; Macdonald et al, Endocrine System, Ch. 56 in Clinical Oncology, M. Abeloffet al, eds., 2nd Ed., 2000.

Role of Histologic Subtype in Thyroid Cancer Prognosis

Prognosis in thyroid carcinoma is related to histologic subtype, degree of differentiation, invasiveness, presence of distant metastases and other factors. In general, more well-differentiated thyroid carcinomas are associated with slower growth, less tissue invasion, fewer distant metastases, and a better prognosis. Poorly differentiated tumors are infiltrative, aggressively metastatic, and associated with a poor prognosis.

There are generally considered to be two types of relatively well-differentiated thyroid carcinomas. Papillary thyroid carcinomas (PTC) are unencapsulated tumors that demonstrate papillary and follicular structures and have distinctive nuclear features (overlapping cell nuclei with the ground glass appearance and longitudinal grooves). Follicular thyroid carcinomas (FTC) are encapsulated tumors with follicular differentiation but lacking PTC's nuclear features. A variety of histologic subtypes of each have been described, and are discussed in Schlumberger et al., New England Journal of Medicine 338: 297-306, 1998.

Two histologic types of poorly differentiated or undifferentiated thyroid carcinoma have also been described. Insular carcinoma is generally thought to be a form of poorly differentiated FTC. It is characterized by oval nests (insulae) of small cells with round nuclei and scant cytoplasm. Growth is infiltrative, and blood vessel invasion is common. The disease is aggressive and often lethal. Anaplastic thyroid carcinoma (ATC) accounts for 5-10% of thyroid carcinomas and is composed of undifferentiated, atypical spindle shaped and multinucleated giant cells. It is highly malignant, rapidly invading adjacent structures and metastasizing throughout the body.

The Problem of Resistance to Therapy

Depending on histologic subtype and extent of disease, thyroid carcinoma is often curable with thyroidectomy and ¹³¹I therapy. However, many histologic subtypes of thyroid carcinoma are inherently resistant to ¹³¹I therapy. This is particularly true of anaplastic and insular thyroid carcinomas, but is also seen in PTC and FTC. In addition, resistance to ¹³¹I may also develop in thyroid carcinomas that at the outset are relatively well-differentiated and relatively sensitive to radioactive iodine.

Although conventional measures are often effective, up to 15% of thyroid cancer subjects will ultimately die from the disease. Thus, new therapeutic approaches to thyroid cancer are needed.

### SUMMARY OF THE DISCLOSURE

A method of enhancing the uptake of an iodide, iodine, or iodine compound in the thyroid is disclosed, by administration of effective amounts of a histone deacetylase inhibitor. In some examples, the iodine or iodide is a radioactive compound that can be administered to treat a thyroid tumor. Novel approaches to thyroid tumor therapy are disclosed herein. Specifically, the use of histone deacetylase inhibitors to threat thyroid neoplasms is disclosed. In another example, a histone deacetylase inhibitor is used to enhance the uptake of iodide for radioprophylaxis following radiation exposure. In one embodiment, a method is provided to enhance thyroid specific gene expression.

Methods are disclosed for detecting thyroid neoplasms in a subject. The method includes administering an effective amount of a histone deacetylase inhibitor and administering a detectable agent whose uptake or concentration in thyroid cells is increased by administration of the histone deacetylase inhibitor. The presence of the detectable agent in the thyroid is then detected.

### BRIEF DESCRIPTION OF THE FIGURES

FIG. 1 is a drawing of the thyroid gland in a human subject (FIG. 1A), and an enlarged view of a thyroid follicle (FIG. 1B) in which thyroid hormone is produced and stored (FIG. 1B). FIG. 1C is an enlarged view of the thyroid follicle shown in FIG. 1B, showing the function of the thyroid follicular cell. Thyroid stimulating hormone (TSH) activates expression of thyroid specific genes and stimulates all of the illustrated processes. The cell takes up iodide (I⁻) and sodium via the Na⁺/I⁻ symporter (NIS). The cell also synthesizes thyroglobulin (TG), and transports it to the follicular lumen. At the luminal surface, iodide is oxidized and enzymatically attached to tyrosine residues on TG. The TG tyrosine residues are enzymatically coupled to form thyroid hormones, in the illustrated case, thyroxine (T4). The modified thyroglobulin is transported from lumen to the apical surface of the follicular cell via endocytosis. In the endolysosomes, TG is degraded to release active T4.

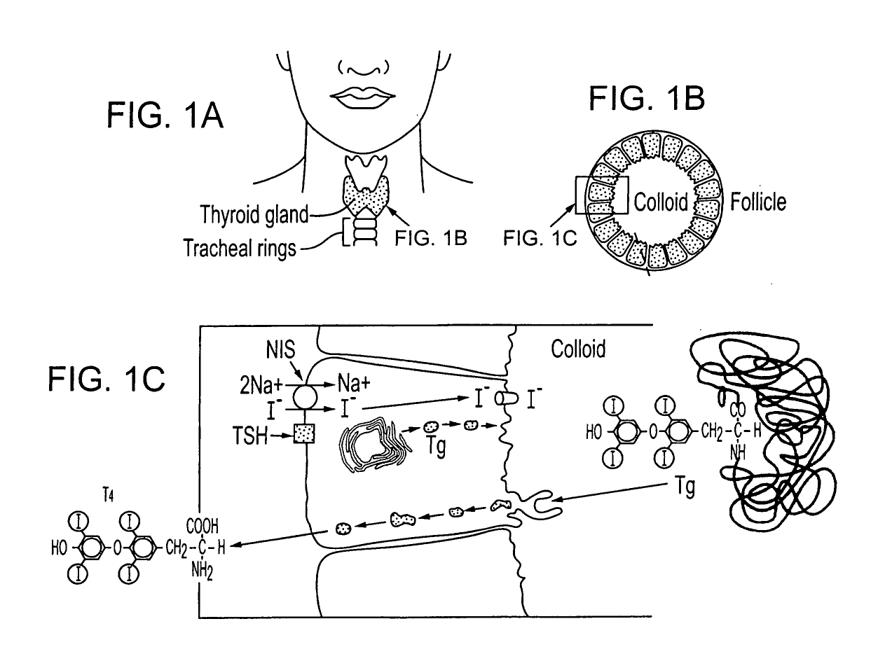

FIG. 2 is a bar graph showing the effect of a histone deacetylase inhibitor on activity of he oglobulin promoter enhancer in thyroid carcinoma cells. Four thyroid carcinoma cell lines were transfected with a plasmid containing the thyroglobulin promoter enhancer element operably linked to a luciferase reporter gene. The transfected cells then either received no treatment, or treatment with the histone deacetylase inhibitor FR901228 (also known as depsipeptide) for 72 hours. Luciferase activity was determined in cell lysates. The effect of FR901228 on luciferase activity was determined, and is plotted as a bar graph of luciferase activity in no-treatment cells (white bars) vs. FR901228-treated cells.

FIG. 3 is a bar graph showing the effect of histone deacetylase inhibition on ability of thyroid carcinoma cells to take up radioactive iodine. FIG. 3A shows ¹²⁵I uptake in four thyroid carcinoma cell lines, and compares uptake in FR901228-treated cells (treated for two or three days as indicated) to uptake in untreated cells. FIG. 3B shows the effect of sodium perchlorate treatment on ¹²⁵I uptake in FR901228-treated thyroid carcinoma cells.

### DETAILED DESCRIPTION

As disclosed herein, histone deacetylase inhibitors affect gene expression in thyroid cancer cells. The effect of histone deacetylase inhibitors can be utilized as a novel approach to thyroid cancer diagnosis and therapy.

Abbreviations Used

DIT dilodotyrosine

MIT monoiodotyrosine

NIS Na⁺/I⁻ symporter

T3 triiodothryronine, a thyroid hormone

T4 thyroxine, a thyroid hormone

TG Thyroglobulin

TPO Thyroid Peroxidase

Explanation of Terms Used

Antisense, Sense, and Antigene: Double-stranded DNA (dsDNA) has two strands, a 5′−>3′ strand, referred to as the plus strand, and a 3′−>5′ strand (the reverse compliment), referred to as the minus strand. Because RNA polymerase adds nucleic acids in a 5′−>3′ direction, the minus strand of the DNA serves as the template for the RNA during transcription. Thus, the RNA formed will have a sequence complementary to the minus strand and identical to the plus strand (except that U is substituted for T).

Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a dsDNA target.

Cancer: A malignant neoplasm that has undergone characteristic anaplasia with loss of differentiation, increase rate of growth, invasion of surrounding tissue, and is capable of metastasis. Thyroid cancer is a malignant neoplasm that arises in or from thyroid tissue. Residual thyroid cancer is thyroid cancer that remains in a subject after any form of treatment given to the subject to reduce or eradicate thyroid cancer. Metastatic thyroid cancer is thyroid cancer at one or more sites in the body other than the site of origin of the original (primary) thyroid cancer from which the metastatic thyroid cancer is derived.

Chemotherapy; chemotherapeutic agents: As used herein, any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating thyroid neoplasms. In one embodiment, a chemotherapeutic agent is radioactive iodine. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

Effective amount of a compound: A quantity of compound sufficient to achieve a desired effect in a subject being treated. For example, a therapeutically effective amount of a compound is the amount necessary to inhibit one or more histone deacetylase isoforms in the cells of a subject, or an amount of a detectable agent administered to detect a neoplasm in a subject.

A therapeutically effective amount of a compound may be administered in a single dose, or in several doses, for example daily, during a course of treatment. However, the therapeutically effective amount of the compound will be dependent on the compound applied, the subject being treated, the severity and type of the affliction, and the manner of administration of the compound.

The general term “administering to the subject” is understood to include administration by any route to all animals (e.g. humans, apes, dogs, cats, horses, and cows) that have or may develop thyroid neoplasms.

Enteral: pertaining to the gastrointestinal tract. In the context of administration of pharmaceutical and/or therapeutic agents, enteral means administration via the gastrointestinal tract, for example by mouth, per rectum, via a nasogastric tube, via a gastrostomy, etc.

Fragments and variants of a polypeptide: This term includes those fragments and variants that maintain one or more biological functions of the parent polypeptide. It is recognized that the gene or cDNA encoding a polypeptide may be considerably mutated without materially altering one or more the polypeptide's biological functions. The genetic code is well-known to be degenerate, and thus different codons encode the same amino acids. Even where an amino acid substitution is introduced, the mutation may be conservative and have no material impact on the essential functions of a protein (e.g. see Stryer, Biochemistry 3rd Ed., (c) 1988). In addition, part of a polypeptide chain can be deleted without impairing or eliminating a function and/or insertions or additions can be made in the polypeptide chain (for example, adding epitope tags) without impairing or eliminating a function (see Ausubel et al., Short Protocols in Molecular Biology, John Wiley & Sons 1998).

Other modifications that can be made without materially impairing one or more functions of a polypeptide include, for example, in vivo or in vitro chemical and biochemical modifications or which incorporate unusual amino acids. Such modifications include, for example, acetylation, carboxylation, phosphorylation, glycosylation, ubiquination, labeling, e.g., with radionuclides, and various enzymatic modifications. A variety of methods for labeling polypeptides and of substituents or labels useful for such purposes are well known in the art, and include radioactive isotopes such as ³²P, ligands which bind to labeled antiligands (e.g., antibodies), fluorophores, chemiluminescent agents, enzymes, and antiligands.

Functional fragments and variants may be of varying length. For example, some fragments have at least about 5, 10, 25, 50, 75,100, 200, 500, 750, or 900 amino acid residues. Such fragments may also have immunogenic activity, and may be used to generate specific binding agent such as antibodies.

Nucleic acid sequences that encode a polypeptide, or a fragment of the polypeptide, can be engineered such that they allow the protein to be expressed in eukaryotic cells, bacteria, insects, and/or plants. In order to accomplish this expression, a regulatory sequences of interest is operably linked to the nucleic acid sequence. The regulatory sequences operably linked to the nucleic acid sequences encoding the polypeptide can be included in a vector. This vector can then be introduced into a host cell. Host cells include, but are not limited to eukaryotic cells, bacterial cells, insect cells, and plant cells.

One of ordinary skill in the art will appreciate that nucleic acid encoding a polypeptide can be altered in numerous ways without affecting the biological activity of the encoded protein. For example, PCR may be used to produce variations in a nucleic acid sequence. Such variants may be variants that are optimized for codon preference in a host cell that is to be used to express the protein, or other sequence changes that facilitate expression.

Two types of cDNA sequence variant may be produced. As noted above, the variation in the cDNA sequence is not manifested as a change in the amino acid sequence of the encoded polypeptide. These silent variations are simply a reflection of the degeneracy of the genetic code. In the second type, the cDNA sequence variation does result in a change in the amino acid sequence of the encoded protein. In such cases, the variant cDNA sequence produces a variant polypeptide sequence. In order to preserve the functional and immunologic identity of the encoded polypeptide, it is preferred that any such amino acid substitutions are conservative. Conservative substitutions replace one amino acid with another amino acid that is similar in size, hydrophobicity, etc. Examples of conservative substitutions are shown in Table 1 below.

TABLE 1

| Original Residue | Conservative Substitution |
|---|---|
| Ala | ser |
| Arg | lys |
| Asn | gln, his |
| Asp | glu |
| Cys | ser |
| Gln | asn |
| Glu | asp |
| Gly | pro |
| His | asn; gln |
| Ile | leu, val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

Variations in the cDNA sequence that result in amino acid changes, whether conservative or not, are ideally minimized in order to preserve the functional and immunologic identity of the encoded protein. Any cDNA sequence variant will preferably introduce no more than 20, and preferably fewer than 10 or 5 amino acid substitutions into the encoded polypeptide. Variant amino acid sequences may, for example, be 80, 90 or even 95% identical to the native amino acid sequence.

In one embodiment, a polypeptide variant is a “dominant negative fragment or variant”, such as a histone deacetylase dominant negative fragment or variant. A histone deacetylase fragment or variant dominant negative variant is a histone deacetylase polypeptide which can bind to one or more histone deacetylase substrates, but which lacks the ability or has impaired ability to deacetylate one or more histone deacetylase substrates. For example, a histone deacetylase fragment or variant can be constructed using recombinant DNA techniques, such that the variant partially or substantially retains binding affinity for one or more histones, but lacks or has impaired ability to deacetylate one or more histones. As a consequence, the dominant negative histone deacetylase fragment or variant acts as a competitive inhibitor of histone deacetylase activity in the cell. In one embodiment, a dominant negative histone deacetylase fragment or variant is a fusion polypeptide.

Fusion protein or polypeptide: A protein or polypeptide encoded by a recombinant nucleic acid that includes two or more amino acid sequences that are not found joined together in nature. A functional fragment or variant of a protein or polypeptide can be included in a fusion protein or polypeptide. In one embodiment, one of the amino acid sequences is an epitope tag. In one specific, non-limiting example, a fusion protein is a dominant negative fragment of histone deacetylase fused to an epitope tag such as green fluorescent protein, FLAG, HisTag, or any number of epitope tags which are known in the art. Convenient cloning vectors (e.g. those produced by Clontech and Promega) are readily available to engineer such fusion proteins, and can be used to express the fusion proteins in a host cell. Without being bound by theory, an epitope tags has little impact on the biological function of the other polypeptide included in the fusion protein, such as protein protein interactions. However, an epitope tag is of use for intracellular location of the fusion protein, purifying the fusion protein, purifying binding partners of the fusion protein, or for immunologic detection.

Histone: A protein that is part of the core proteins of nucleosomes. Acetylation and deacetylation of histones play a role in regulating gene expression, by affecting the structure of nucleosomes and chromatin. Histone proteins include, but are not limited to, H2A, H2B, H3, and H4. Histones can be in two forms, acetylated and deacetylated. Histone acetyltransferase causes the acetylation of histones, while histone deacetylase reverses this process. Deacetylation of histones involves the removal, through hydrolysis, of an acetyl group form the ε-amino group of the histone's lysine side chains.

Histone deacetylase: A class of enzymes, also called protein deacetylases, that catalyze removal of an acetyl group from the epsilon-amino group of lysine side chains in histones or other proteins (for example, histones H2A, H2B, H3 or H4), thereby reconstituting a positive charge on the lysine side chain (Ng and Bird, Rends in Biol. Sci. 25:121-136, 2000, herein incorporated by reference). See also Emiliani et al., Proc. Natl. Acad. Sci. U.S.A. 95: 2795-2800, 1998; Fischle et al., J. Biol. Chem. 274: 11713-11720, 1999; Yang et al. Proc. Natl. Acad. Sci. U.S.A. 93: 12845-12850, 1996; Taunton et al., Science 272: 408-411, 1996). Several histone deacetylases have been identified, including, but not limited to HDAC1, HDAC2, and RPD3. Specific, non-limiting examples of a histone deacetylase include, but are not limited to, GenBank Accession Nos. NM 058277, NM15401, AF407273, XM 004379, and AF 426160, AF006603, AF006602,and AF074882, see also U.S. Pat. No. 6,287,843. When histone deacetylase is inhibited, the activity of the counter enzyme, histone acetyltransferase, is in relative excess, and hyperacetylation of histones or other proteins occurs. Without being bound by theory, inhibition of histone deacetylase results in the lysine tails of histones becoming neutralized, disruption of the histone structure, and unfolding of DNA. The unfolded state of the histone permits transcription factors to access the DNA.

Histone deacetylase inhibitor: An agent that inhibits the function of one or more histone deacetylases, for example by 10%, 20%, 30%, 40%, 50%,80%, 95% or more. Such agents may take the form of a pharmaceutical agent or drug, a therapeutically effective oligonucleotide, a specific binding agent, or a fragment or variant of histone deacetylase. Several structural classess of histone deacetylase inhibitors have been identified. These include (1) short-chain fatty acids (for example butyrates), (2) hydroxamic acids (for example suberic bishydroxmic acid, suberolylanilde hydroxamic acid, proxamide, m-carboxy cinnamic acid bishydroxamic acid), (3) cyclic tetrapeptides containing 2-amino8-oxo-9,10-epoxy-decanoyl moiety, and (4) benzamides. Specific, non-limiting examples of a histone deacetylase inhibitor include FR901228 (depsipeptide), scriptaid, N-acetyldinaline (CI-994), Scriptaid, suberoylanilide hydroxamic acid, trichostatin A, trapoxin A, trapoxin B, HC-toxin, chlamydocin, Cly-2, WF-3161, Tan-1746, apicidin, analogs of apicidin, benzamide, derivatives of benzamide, hydroxyamic acid derivatives, azelaic bishydroxyamic acid, butyric acid and salts thereof, actetate salts, suberoylanilide hydroxyamide acid, suberic bishydroxyamic acid, m-carboxy-cinnamic acid bishyrdoxyamic acid, oxamflatin, depudecin, or MS-275 (Marks et al., Curr. Opinion in Oncol. 13:477, 2001, herein incorporated by reference in its entirety). Alternatively, the agent may be a therapeutically effective oligonucleotide that inhibits expression or function of histone deacetylase, such as an antisense molecule or a ribozyme. Alternatively, a histone deacetylase inhibitor can be a dominant negative fragment or variant of histone deacetylase.

Injectable composition: A pharmaceutically acceptable fluid composition comprising at least one active ingredient, e.g. a bispecific fusion protein. The active ingredient is usually dissolved or suspended in a physiologically acceptable carrier, and the composition can additionally comprise minor amounts of one or more non-toxic auxiliary substances, such as emulsifying agents, preservatives, and pH buffering agents and the like. Such injectable compositions that are useful for use with the fusion proteins of this invention are conventional; appropriate formulations are well known in the art.

Neoplasm: An abnormal cellular proliferation, which includes begnin and malignant tumors, as well as other proliferative disorders.

Open reading frame: A series of nucleotide triplets (codons) coding for amino acids without any internal termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, are in the same reading frame.

Na⁺/I⁻ symporter (sodium/iodide symporter, NIS): A membrane protein that mediates active transport of iodide anion into the thyroid. See, for example, De la Vieja et al., Physiological Reviews 80: 1083-1105, 2000; Dai et al., Nature 379: 458-460, 1966. Also referred to herein as the sodium-iodide symporter.

Parenteral: Administered outside of the intestine, e.g., not via the alimentary tract. Generally, parenteral formulations are those that will be administered through any possible mode except ingestion. This term especially refers to injections, whether administered intravenously, intrathecally, intramuscularly, intraperitoneally, or subcutaneously, and various surface applications including intranasal, intradermal, and topical application, for instance.

Pharmaceutical agent or drug: A chemical compound or composition capable of inducing a desired therapeutic or prophylactic effect when a specified dose is administered to a subject.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: A promoter is an array of nucleic acid control sequences that directs transcription of a nucleic acid. A promoter includes necessary nucleic acid sequences near the start site of transcription, such as, in the case of a polymerase II type promoter, a TATA element. A promoter also optionally includes an enhancer or a repressor element that can be located as much as several thousand base pairs from the start site of transcription.

Purified: The term “purified” does not require absolute purity; rather, it is intended as a relative term. Thus, for example, a purified protein preparation is one in which the protein referred to is more pure than the protein in its natural environment within a cell.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques.

Specific binding agent: An agent that specifically binds only to a defined target. Thus a histone deacetylase-specific binding agent binds substantially only a histone deacetylase isoform. As used herein, the term “histone deacetylase specific binding agent” includes histone deacetylase protein antibodies and other agents that bind substantially only to histone deacetylase.

Anti-histone deacetylase protein antibodies may be produced using standard procedures described in a number of texts, including Harlow and Lane (Antibodies, A Laboratory Manual, CSHL, New York, 1988). The determination that a particular agent binds substantially only to a histone deacetylase may readily be made by using or adapting routine procedures. One suitable in vitro assay makes use of the Western blotting procedure (described in many standard texts, including Harlow and Lane, Antibodies, A Laboratory Manual, CSHL, New York, 1988; Ausubel et al., in Molecular Biology, CSHL, New York, 1998).

Shorter fragments of antibodies can also serve as specific binding agents. For instance, Fabs, Fvs, and single-chain Fvs (SCFvs) that bind to a histone deacetylase would be histone deacetylase-specific binding agents. These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)2, the fragment of the antibody obtained by treating whole antibody with the enzyme pepsin without subsequent reduction; (4) F(ab′)2, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule.

Therapeutically effective dose: A dose sufficient to prevent advancement, or to cause regression of the disease, or which is capable of relieving symptoms caused by the disease, such as fever, pain, decreased appetite or cachexia associated with malignancy.

Therapeutically Effective Oligonucleotides and Oligonucleotide Analogs: Therapeutically effective oligonucleotides and oligonucleotide analogs are characterized by their ability to inhibit a function of a protein, for example by inhibiting the expression of a protein. Inhibition can be any reduction in target protein activity or expression seen when compared to target protein activity or expression in the absence of the oligonucleotide or oligonucleotide analog. Additionally, some oligonucleotides will be capable of inhibiting the activity or expression of a target protein by at least 15%, 30%, 40%, 50%, 60%, or 70%, or more.

Some therapeutically effective oligonucleotides and oligonucleotide analogs are additionally characterized by being sufficiently complementary to target protein-encoding nucleic acid sequences. As described herein, sufficiently complementary means that the therapeutically effective oligonucleotide or oligonucleotide analog can specifically disrupt the expression of the target protein, and not significantly alter the expression of genes other the target nucleic acid sequence.

For example, a therapeutically effective oligonucleotide may reduce histone deacetylase activity in a cell by at least 15%, 30%, 40%, 50%, 60%, or 70%, or more.

The term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

Thyroglobulin: A large (about 2750 amino acid residues) iodoglycoprotein which is specifically expressed in thyroid tissue and is the substrate for the synthesis of thyroid hormones, thyroxine and triiodothyronine. See Malthiery et al., Biochimie 71: 195-209, 1989; Vassart et al, Molecular and Cellular Endocrinology 30: 89-97, 1985; van de Graafet al, European Journal of Endocrinology 136: 508-515, 1997.

Thyroid Peroxidase (TPO): A thyroid-specific enzyme that catalyzes the oxidation and organification of iodide anion in follicular cells. See Ohtaki et al, Endocrine Journal. 43: 1-14, 1996

Thyroid specific gene expression: Genes which are expressed at high levels in thyroid follicular cells, relative to non-thyroid cell types (for example, at least five fold higher than in non-thyroid cell types). These include, for example, thyroglobulin, the Na⁺/I⁻ symporter, and thyroid perioxidase. The terms “thyroid-specific,” “specifically expressed,” or the like, do not imply that these genes are not expressed in any other cell type. For example, it is known that the Na⁺/I⁻ symporter is expressed in lactating mammary cells, salivary gland cells, and gastric mucosa. Specific, non-limiting examples of thyroid specific genes are thyroglobulin and/or the Na⁺/I⁻ symporter in thyroid cancer cells. Without being bound by theory, enhanced expression of thyroid-specific, genes promotes cellular differentiation and reduces biologically aggressive behavior such as invasion and metastasis. In addition, without being bound by theory, enhanced expression of thyroglobulin and/or the Na⁺/I⁻ symporter increases the ability of thyroid cancer cells to concentrate iodine, thereby making the cells more susceptible to radioactive iodine therapy.

Tumor: A neoplasm that may be either malignant (cancerous) or non-malignant.

Unless otherwise explained, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and are not intended to be limiting.

### Overview of Some Disclosed Methods

A method is provided herein for inhibiting the growth of a thyroid cancer cell. The method includes administering a therapeutically effective amount of a histone deacetylase inhibitor. As disclosed herein, inhibition of histone deacetylases increase transcriptional activity of the thyroid-specific thyroglobulin promoter-enhancer element, and also activates transcription of thyroid-specific genes. Specific, non-limiting examples of genes showing increased expression include the Na⁺/I⁻ symporter (NIS) and thyroglobulin, two proteins that participate in iodide transport, iodine concentration, and thyroid hormone production in thyroid cells. Without being bound by theory, the increased expression of thyroid specific genes induced by histone deaceiylase inhibitors is believed to promote differentiation of thyroid cancer cells, thereby reducing biologically aggressive behavior such as invasion and metastasis. In addition, increased expression of thyroid specific genes enhances or restores ability to concentrate iodide, thereby rendering thyroid cancer cells susceptible to treatment with radioactive iodine therapy.

Disclosed herein are methods that enhance expression of thyroid specific genes in thyroid cancer cells, by introducing into the thyroid cancer cell an agent that inhibits at least one histone deacetylase. In one embodiment, a method is provided for enhancing expression of a gene comprising a thyroglobulin promoter-enhancer element, including the thyroglobin gene including the thyroglobulin encoding sequence, or a gene comprising the throglobulin promoter operably linked to a heterologous nucleic acid sequence. In another embodiment, a method is provided for enhancing expression of Na⁺/I⁻ symporter or a gene comprising the Na⁺/I⁻ symporter promoter operably linked to a heterlogous nucleic acid sequence. In a further embodiment, a method is provided for enhancing the ability of thyroid cancer cells to take up or concentrate iodide or iodine. In yet another embodiment, a method is provided for enhancing the ability of a thyroid cell to take up iodide or iodine, such as KI.

The agent that inhibits a histone deacetylase can be, for example, a therapeutically effective amount of FR901228 (depsipeptide), trichostatin A, trapoxin A, trapoxin B, HC-toxin, chlamydocin, Cly-2, WF-3161, Tan-1746, apicidin, analogs of apicidin, benzamide, derivatives of benzamide, hydroxyamic acid derivatives, azelaic bishydroxyamic acid, butyric acid and salts thereof, actetate salts, suberoylanilide hydroxyamide acid, suberic bishydroxyamic acid, m-carboxy-cinnamic acid bishyrdoxyamic acid, oxamflatin, depudecin, or MS-27-275. Alternatively, the agent can be a therapeutically effective oligonucleotide that inhibits expression or function of histone deacetylase, or a dominant negative fragment or variant of histone deacetylase. Agents that inhibit histone deacetylase have also been described in WO0071703A2, WO017675A2, and WO0008048A2.

The thyroid cancer cell can be a thyroid cancer cell in a subject, and the method can be a method of treating the thyroid cancer by administering a therapeutically effective amount of radioactive iodine, for example ¹³¹I, to the subject. The therapeutically effective amount of ¹³¹I can be, for example, from about 1 mCi to about 500 mCi, or from about 30 mCi to about 300 mCi. In specific embodiments, the thyroid cancer cell in the subject can be derived from a papillary thyroid carcinoma, a follicular thyroid carcinoma, an insular thyroid carcinoma, an anaplastic thyroid carcinoma, or any histologic variant of thyroid carcinoma.

In other specific embodiments, treatment with radioactive iodine can be accompanied by additional treatments, such as thyroidectomy, external beam irradiation, or administration of a therapeutically effective amount of an anticancer chemotherapeutic agent to the subject. In still other specific embodiments, radioactive iodine can be administered to the subject a plurality of times, for example on 2, 3, 4, 5, 6, 7, 8, 9, 10, or more occasions. These occasions can be separated by a period of time, for example more than about twelve hours, more than about 24 hours, more than about 48 hours, more than about 72 hours, more than about one week, more than about two weeks, more than about four weeks, more than about eight weeks, more than about twelve weeks, more than about six months, more than about one year, or more than about two years. On any one or more of these occasions, the administration of a histone deacetylase inhibitor and/or radioactive iodine can be accompanied by administration of a therapeutically effective amount of thyroid stimulating hormone.

Also disclosed are methods of detecting a thyroid neoplasm in a subject, by administering to the subject a therapeutically effective amount of a histone deacetylase inhibitor; administering to the subject a detectable agent whose concentration in thyroid carcinoma cells is increased by administration of the histone deacetylase inhibitor; and detecting the detectable agent. The method includes detecting a thyroid neoplasm by detecting the agent whose concentration in neoplastic thyroid cells is increased by administration of the histone deacetylase inhibitor. The agent can be an agent that is transported into thyroid cells via the Na⁺/I⁻ symporter. The agent can be, for example, a radioactive iodide molecule, for example, ¹²³I, ¹²⁵I, or ¹³¹I; or a radiolabeled perchlorate or pertechnitate.

In specific embodiments, the method can be a method of detecting papillary, follicular, insular, or anaplastic thyroid carcinoma. In other specific embodiments, the method can be a method of detecting residual thyroid carcinoma in a subject who has received one or more therapies to treat thyroid carcinoma. For example, the method can be a method of detecting residual thyroid carcinoma in a subject who has had thyroidectomy, ¹³¹I, external irradiation, or who has received an anticancer chemotherapeutic agent.

In yet other embodiments, the iodide or iodine compound is administered prophylatically following exposure to radiation (for example following a nuclear plant accident or exposure to a nuclear weapon) to enhance the effectiveness of radiophophlyaxis against the development of thyroid neoplasms.

### Examples of Histone Deacetylase Inhibitors

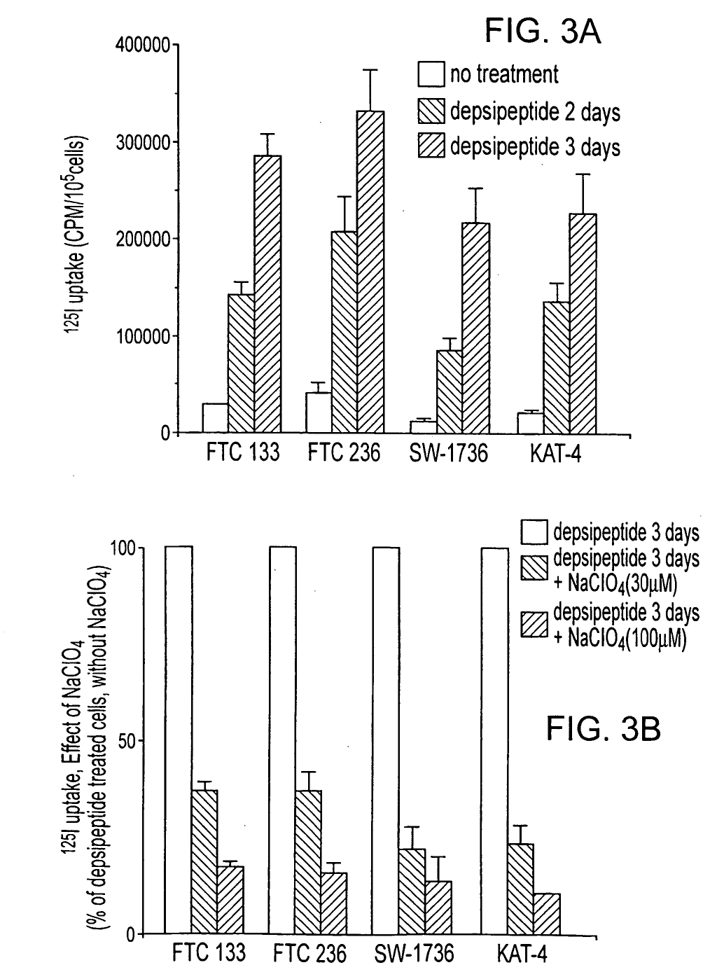

Marks et al., Journal of the National Cancer Institute 92: 1210-1216, 2000, incorporated by reference herein, gives an overview of types and classes of histone deacetylase inhibitors. All of the histone deacetylase inhibitors described in Marks et al. are useful in the methods of the present disclosure. See, for example, the HDIs described in FIGS. 3 and 4 of Marks et al. (pages 1212-1213).

Cyclic tetrapeptides such as those described in PCT publications WO 00/08048 and WO 00/21979, and U.S. Pat. No. 5,922,837 can be used in the present invention. FR901228 and related compounds, disclosed in U.S. Pat. No. 4,977,138, are also suitable. Other useful HDIs include sodium butyrate, trichostatin A, trapoxin A, trapoxin B, HC-toxin, chlamydocin, Cly-2, WF-3161, Tan-1746, and apicidin and analogs thereof.

HC-Toxin is described in Liesch et al. (1982) Tetrahedron 38, 45-48; Trapoxin A and Trapoxin B are described in Itazaki et al. (1990) J. Antibiot. 43, 1524-1532 and EP0406725; WF-3161 is described in Umehana et al. (1983) J. Antibiot. 36, 478-483; Cly-2 is described in Hirota et al (1973) Agri. Biol. Chem 37, 955-56; Chlamydocin is described in Closse et al. (1974) Helv. Chim. Acta 57, 533-545 and Tan 1746 is described in Japanese Patent No. 7196686 to Takeda Yakuhin Kogyo KK. Benzamide and derivatives are described in Suzuki et al., Journal of Medicinal Chemistry 42: 3001-3003, 1999, and JP11335375.

Hydroxyamic acid derivatives are useful in the methods of the present disclosure. Examples include azelaic bishydroxyamic acid, described in Qiu et al., Molecular Biology of the Cell 11: 2069-2083, 2000; and suberoylanilide hydroxyamide acid, described in Richon et al., Proc. Natl. Acad. Sci. U.S.A. 97: 10014-10019, 2000.

Thus, specific, non-limiting examples of a histone deacetylase inhibitor include, but are not limited to, FR901228 (depsipeptide), trichostatin A, trapoxin A, trapoxin B, HC-toxin, chlamydocin, Cly-2, WF-3161, Tan-1746, apicidin, analogs of apicidin, benzamide, derivatives of benzamide, hydroxyamic acid derivatives, azelaic bishydroxyamic acid, butyric acid and salts thereof, actetate salts, suberoylanilide hydroxyamide acid, suberic bishydroxyamic acid, m-carboxy-cinnamic acid bishyrdoxyamic acid, oxamflatin, depudecin, or MS-27-275.

Specific binding agents can also be histone deacetylase inhibitors, for example antibodies and/or antibody fragments that specifically bind to histone deacetylase and inhibit its function (see above).

Therapeutically effective oligonucleotides can also be used as histone deacetylase inhibitors. In one specific, non-limiting example, the oligonucleotide is an antisense oligonucleotide that inhibits expression of histone deacetylase. Use of therapeutically effective oligonucleotides are further described in Example 6.

Fragments and variants of histone deacetylase also may be used as histone deacetylase inhibitors. In one specific non-limiting example, the histone deacetylase inhibitor is a dominant negative variant of histone deacetylase. Human histone deacetylase A is known to have a catalytically active domain from about amino acid residue 490 through the C-terminal amino acid residue 967 (Fischle et al., J. Biol. Chem. 274: 11713-11720, 1999). In addition, it is known that a region from about residue 495 through about residue 550 is essential for histone deacetylase catalytic activity. Thus, a human histone deacetylase A fragment encompassing residues 540 through the C-terminal residue 967 would retain ability to bind histone substrates, but lack the ability to deacetylate the substrate. Thus, this fragment is a dominant negative fragment of histone deacetylase, and can be used as a histone deacetylase inhibitor. Additional dominant negative histone deacetylase fragments are readily designed by sequence homology searches, and constructed using standard DNA recombinant techniques such as those described in Ausubel et al., Short Protocols in Molecular Biology, John Wiley & Sons, 1998.

Dominant negative histone deacetylase fragments or variants inhibit histone deacetylase in thyroid cells. Such fragments may be delivered to neoplastic thyroid cells in a variety of manners. For example, a nucleic acid encoding a dominant negative histone deacetylase fragment may be transferred to an appropriate eukaryotic gene transfer vector and delivered to tumor cells.

Viral vectors, such as retroviral vectors, are of use for eukaryotic gene transfer, with a high efficiency of infection and stable integration and expression (Orkin et al., Prog. Med. Genet. 7:130-142, 1988). A nucleic acid encoding a dominant negative histone deacetylase fragment or variant can be cloned into a retroviral vector and driven from either its endogenous promoter, a heterologous promoter (constitutive or inducible) or from the retroviral LTR (long terminal repeat). Other viral transfection systems may also be utilized for this type of approach, including adenovirus, adeno-associated virus (AAV) (McLaughlin et al., J. Virol. 62:1963-1973, 1988), Vaccinia virus (Moss et al., Annu. Rev. Immunol. 5:305-324, 1987), Bovine Papilloma virus (Rasmussen et al., Methods Enzymol. 139:642-654, 1987), members of the herpesvirus group such as Epstein-Barr virus (Margolskee et al., Mol. Cell. Biol. 8:2837-2847, 1988), or lentivirus and related vectors (U.S. Pat. No. 6,013,516).

Very large nucleic acid inserts can be integrated into viral systems. Kochanek et al., Proc. Natl. Acad. Sci. USA 93: 5731-5739, 1996, have demonstrated efficient packaging in an adenoviral system of a 28.2 kb expression cassette for use in gene transfer therapy. Also working in adenoviruses, Parks and Graham have demonstrated packaging of vectors with sizes ranging from 15.1 to 33.6 kb (Parks et al., Proc. Natl. Acad. Sci. USA 93:13565-13570, 1996; Parks and Graham, J. Virol. 71:3293-3298, 1997).

In one embodiment, adenovirus-mediated gene delivery is used to direct expression of a nucleic acid in thyroid cells. Blagosklonny et al., Journal of Clinical Endocrinology & Metabolism. 83(7):2516-22, 1998 demonstrated that adenovirus-mediated gene transfer was highly effective in restoring functional p53 status to anaplastic thyroid carcinoma cells. Zeiger et al., Surgery 120: 921-925, 1996, demonstrated the efficacy of adenovirus-mediated thymidine kinase gene transfer in promoting tumor cell kill by ganciclovir administration.

Recent developments in eukaryotic gene transfer techniques include the use of RNA-DNA hybrid oligonucleotides, as described by Cole-Strauss, et al. (Science 273:1386-1389, 1996). This technique may allow for site-specific integration of cloned sequences, thereby permitting accurately targeted gene modification.

It is possible to use non-infectious methods of delivery. For instance, lipidic and liposome-mediated gene delivery can be used for transfection with various genes (for reviews, see Templeton and Lasic, Mol. Biotechnol. 11:175-180, 1999; Lee and Huang, Crit. Rev. Ther. Drug Carrier Syst. 14:173-206; and Cooper, Semin. Oncol. 23:172-187, 1996), and for delivery of peptides. For instance, cationic liposomes have been analyzed for their ability to transfect monocytic leukemia cells, and shown to be a viable alternative to using viral vectors (de Lima et al., Mol. Membr. Biol. 16:103-109, 1999). Such cationic liposomes can also be targeted to specific cells through the inclusion of, for instance, monoclonal antibodies or other appropriate targeting ligands (Kao et al., Cancer Gene Ther. 3:250-256, 1996). Sikes et al., Human Gene Therapy 5:837-844, 1994, successfully used direct interstitial injection of plasmid DNA into the thyroid gland to transform thyroid follicular cells.

Thus, a variety of techniques are available to introduce dominant negative histone deacetylase fragments and variants into neoplastic thyroid cells.

### Examples of the Therapeutic Use of Histone Deacetylase Inhibitors

For treatment of thyroid neoplasms in humans or animals, a histone deacetylase inhibitor (HDI) is administered once the disease is diagnosed. Treatment may be intermittent or continuous. An example of intermittent therapy is administration of a therapeutically effective amount of an HDI for a short time prior to administration of ¹³¹I, as described in Example 5 and references therein. For example, a therapeutically effective amount of an HDI could be administered for about 12 hours, about 24 hours, about 48 hours, about 72 hours, about five days, about one week, about two weeks, or about four weeks prior to ¹³¹I administration. The daily dose would vary between about 0.01 μg/kg to about 500 mg/kg. Treatments can be one or more time(s) a day, enterally or parenterally, until a therapeutically effective amount of an HDI is given, or until maximum therapeutic HDI inhibition is obtained. Alternatively, the daily dose can be administered approximately continuously, for example by intravenous infusion.

A HDI may also be given to promote differentiation of thyroid cancer cells, thereby reducing their tendency to biologically aggressive behavior such as tissue invasion and metastasis. Therapy may be intermittent or continuous, as described above. One example of continuous therapy to promote differentiation of thyroid cancer cells would be a daily dose between about 0.01 μg/kg to about 500 mg/kg, given until a favorable therapeutic response is observed, or until all thyroid cancer cells in a subject are eliminated or killed. Such differentiation-promoting HDI therapy can be given regardless of whether radioactive iodine or other thyroid cancer therapy is also given.

In some instances, a therapeutically effective amount of an HDI can be administered to a subject who has undergone, or who will undergo, a thyroidectomy. The administration of HDI to a subject who has undergone (or will undergo) thyroidectomy is not materially different than in subjects who have not undergone thyroidectomy.

Histone deacetylase inhibitors can be administered enterally or parenterally to a subject in need of treatment. The dosage to be administered may vary according to the particular compound used, the histologic type of thyroid cancer involved, the particular host, the severity and extent of the disease, physical condition of the host, and the selected route of administration. The appropriate dosage can be readily determined by a person skilled in the art. For example, for the treatment of thyroid carcinoma in human and animals, the daily dosage may range from about 0.01 μg/kg to about 500 mg/kg.

The compositions disclosed herein include a histone deacetylase inhibitor and an inert carrier. The compositions can be in the form of pharmaceutical compositions for human and veterinary usage, or in the form of feed composition for the control of coccidiosis in poultry. The term “composition” is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions of one or more of the ingredients. The composition thus includes a composition when made by admixing a histone deacetylase inhibitor and inert carrier.

The pharmaceutical compositions include a histone deacetylase inhibitor as an active ingredient, and can also contain a pharmaceutically acceptable carrier and optionally other therapeutic ingredients, such as other chemotherapeutic agents. The compositions include compositions suitable for oral, rectal, topical, and parenteral (including subcutaneous, intramuscular, and intravenous) administrations, although the most suitable route in any given case will depend on the particular host, and nature and severity of the conditions for which the active ingredient is being administered. The pharmaceutical compositions may be conveniently presented in unit dosage form and prepared by any of the methods well-known in the art of pharmacy.

In practical use, a histone deacetylase inhibitor can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous).

In preparing the compositions for oral dosage form, any of the usual pharmaceutical media can be employed. For example, in the case of oral liquid preparations such as suspensions, elixirs and solutions, water, glycols, oils, alcohols, flavoring agents, preservatives, coloring agents and the like can be used; or in the case of oral solid preparations such as powders, capsules and tablets, carriers such as starches, sugars, microcrystalline cellulose, diluents, granulating agents, lubricants, binders, disintegrating agents, and the like can be included. Because of their ease of administration, tablets and capsules represent the most advantageous oral dosage unit form in which case solid pharmaceutical carriers are obviously employed. If desired, tablets can be coated by standard aqueous or nonaqueous techniques. In addition to the common dosage forms set out above, histone deacetylase inhibitors can also be administered by controlled release means and/or delivery devices.

Pharmaceutical compositions suitable for oral administration can be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions can be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

Pharmaceutical compositions of the present invention suitable for parenteral administration can, for example, be prepared as solutions or suspensions of these active compounds in water suitably mixed with a surfactant such as hydroxypropylcellulose. Dispersions can also be prepared in glycerol, liquid polyethylene glycols, and mixtures thereof in oils. Under ordinary conditions of storage and use, these preparations contain a preservative to prevent the growth of microorganisms.

Examples of pharmaceutical forms suitable for injectable use include sterile aqueous solutions or dispersions and sterile powders for the extemporaneous preparation of sterile injectable solutions or dispersions. In all cases, the form should be sterile and fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

It should be understood that in addition to the aforementioned carrier ingredients the pharmaceutical formulations described above may include, as appropriate, one or more additional carrier ingredients such as diluents, buffers, flavoring agents, binders, surface-active agents, thickeners, lubricants, preservatives (including anti-oxidants) and the like, and substances included for the purpose of rendering the formulation isotonic with the blood of the intended recipient.

### Diagnostic Use of Histone Deacetylase Inhibitors

Histone deacetylase inhibitors can be administered enterally or parenterally to a subject known to have, or suspected of having a thyroid neoplasm. Administration of histone deacetylase inhibitors can aid in the diagnosis of thyroid neoplasm, for example by allowing the determination of the presence and extent of a thyroid neoplasm. Administration of histone deacetylase inhibitors can also enhance the sensitivity, specificity, or predictive value of diagnostic tests for thyroid neoplasms.

Localization of functioning or nonfunctioning thyroid tissue in the area of the thyroid gland or elsewhere is made possible by techniques of external scintiscanning. The underlying principle is that isotopes that are differentially accumulated by thyroid tissue can be detected and quantified in situ. The data can be digitized and transformed into a visual display. Such accumulated isotopes may be detected by an external scintillation detector, as described in Larsen et al., The Thyroid Gland, Chapter 11 in Williams' Textbook of Endocrinology, J. Wilson editor, 1998.

Thyroid imaging agents are generally taken up into the thyroid via thyroid-specific genes, such as the Na⁺/I⁻ symporter. Thus, the ability of histone deacetylase inhibitors to increase the expression of thyroid specific genes, for example the Na⁺/I⁻ symporter and/or TG, enhances the ability of thyroid cells to take up and/or retain such imaging agents. Thus, histone deacetylase inhibition makes thyroid tissue more readily detectable externally, for example by external scintiscanning.

Administration of histone deacetylase inhibitors can be particularly useful in a variety of common clinical circumstances. For example, poorly differentiated thyroid carcinomas are often difficult to detect because they have lost the ability to concentrate diagnostic agents. Without being bound by theory, it is likely that this is related to their reduced expression of thyroid specific genes such as Na⁺/I⁻ symporter and/or thyroglobulin. Administration of histone deacetylase inhibitors enhances expression of thyroid specific genes in poorly differentiated thyroid carcinomas, thereby enhancing uptake of diagnostic agents and making the thyroid carcinoma more readily detectable. Thus, the sensitivity and positive predictive value of such tests are improved, making their results more clinically useful. See Goldman, Quantitative Aspects of Clinical Reasoning, Chapter 3 in Harrison's Principles of Internal Medicine, 14th ed., A. Fauci Ed., copyright 1998.

Problems with detection of thyroid carcinoma also can occur, for example, following therapy with thyroidectomy, radioactive iodine, anticancer chemotherapeutic agents, or external radiation.

Several radioisotopes are employed in thyroid imaging. ^{99m}Tc-pertechnetate (TcO⁻₄) is a monovalent anion that, like iodide, is actively concentrated in the thyroid gland via the Na⁺/I⁻ symporter. The short physical half-life of ^{99m}Tc (6 h) and its transient stay within the thyroid make the radiation delivered to the thyroid by a standard dose very low. Consequently, the administration of large doses (>37 MBq [1 mCi]) permits high counting rates and adequate imaging of the thyroid when the fractional uptake is too low to permit scintiscanning with radioiodine. Pertechnetate is usually given as a single intravenous bolus, and imaging is performed about 30 min later. Serial imaging makes possible studies of the dynamics of thyroid blood flow and isotope accumulation.

Three radioactive isotopes of iodine have been used in thyroid imaging. ¹³¹I was commonly used in the past, and it is still useful when functioning metastases of thyroid carcinoma are being sought. The physical half-life of ¹²⁵I (60 d) is longer than that of ¹³¹I (8 d), but its lower radiation energy results in the delivery of a radiation dose to the thyroid per unit of radioactivity administered that is only about two thirds that delivered by ¹³¹I . The third isotope, ¹²³I, is better in many respects than ¹²⁵I or ¹³¹I . Its short half-life and the absence of beta radiation result in a radiation dose to the thyroid that is about 1% of that delivered by a comparable dose of ¹³¹I . All three isotopes of iodine provide satisfactory images of the thyroid in its normal location. Because of the low radiation dose to the thyroid, ¹²³I is useful for thyroid scintigraphy in human pediatric practice.

### EXAMPLE 1

### Effects of a Histone Deacetylase Inhibitor on Cell Viability

This example demonstrates that FR901228, a hsitone deacetylase inhibitor, is not significantly cytotoxic. In particular, exposure to up to 1 ng/ml FR901228 for 72 hours was not significantly cytotoxic to four different thyroid carcinoma cell lines.

Methods and Materials

FTC 133 and FTC 236 cells were derived from cultures obtained from the primary tumor (FTC 133) and a nodal metastasis (FTC 236) of a human follicular thyroid carcinoma. FTC 133 and FTC 236 cells were originally maintained in medium containing TSH, but this was discontinued after it was determined that TSH had no effect on growth rates in the cells. Anaplastic thyroid carcinoma lines SW-1736 and KAT-4 were derived from primary cultures of human anaplastic thyroid carcinoma tumors.

Each of these four cell lines was exposed to varying concentrations of FR901228 (0.01 ng/ml, 0.1 ng/ml, 1 ng/ml, 10 ng/ml) for 72 hours, following which cell viability was determined using the 3-[4,5-dimethylthiazol-2-yl]-2,5-diphenyl tetrazoliumbromide (MTT) assay, as described in Mosmann, J Immunol Methods 65:55-63, 1983, and Den Boer et al., Br. J. Haem. 105: 876-882, 2000. The MTT assay is a colorimetric assay of the loss of mitochondrial metabolic activity associated with cell death.

Results and Conclusions

This example demonstrated that exposure to a FR901228 concentration of 1 ng/ml for 72 hours was not significantly cytotoxic for any of the four cell types tested (FTC 133, FTC 236, SW-1736 and KAT-4). Thus, subsequent methods to evaluate cellular FR901228 effects could be safely performed at 1 ng/ml FR901228, without confounding the results by FR901228-induced cytotoxicity. Accordingly, methods described in the subsequent examples were performed at 1 ng/ml FR901228, unless otherwise indicated.

### EXAMPLE 2

### FR901228 Inhibits Histone Deacetylase in Thyroid Carcinoma Cell Lines

To demonstrate that FR901228 was effective in inhibiting histone acetylation, the extent of histone acetylation was evaluated in FR901228- and control-treated thyroid carcinoma cells. These immunofluorescence studies showed that treatment with 1 ng/ml FR901228 for 72 hours resulted in a marked increase in histone acetylation in both more differentiated (FTC 236) and less differentiated (SW-1736) thyroid carcinoma cells. Thus, 1 ng/ml FR901228 effectively inhibits histone deacetylation in thyroid carcinoma cells, without causing significant cytotoxicity.

Methods and Materials

Histone acetylation was detected by immunofluorescence microscopy. In this experimental approach, fixed cells are exposed to a primary antibody that specifically binds to acetylated histones, and then exposed to an FITC-labeled secondary antibody that binds to the primary antibody. Cells containing acetylated histones demonstrate nuclear fluorescence that reflects the presence of acetylated histones.

FTC 236 and SW-1736 cells were cultured as described in Example 1, and treated for 72 hours with 1 ng/ml FR901228. Control FTC 236 and SW-1736 received no FR901228, but were otherwise treated identically to the FR901228-exposed cells. After 72 hours, each culture was treated with trypsin, harvested, and subjected to low speed centrifugation to form a cell pellet. Cells from each pellet were placed on microscope slides and fixed with 95% ethanol/5% acetic acid for one minute at room temperature. After fixation, slides were washed twice with phosphate buffered saline (PBS) for 15 minutes, treated with 8% bovine serum albumin in PBS for one hour at room temperature, and washed 15 minutes in PBS.

The fixed cells were then incubated overnight at 4° C. with 5 ug/ml anti-alpha acetylated histone H3 in 2% bovine serum albumin in PBS (antibody obtained from Upstate Biotechnology, Lake Placid N.Y.). Subsequently, cells were washed twice with PBS for five minutes at room temperature and then incubated with horse anti-rabbit FITC-conjugated secondary antibody (from Vector Labs, Burlingame Calif.). Slides were then washed three times with PBS for 15 minutes and then counterstained with DAPI-containing antifade compound (from Vector Labs, Burlingame Calif.).

Results and Conclusions

Examination of control cells revealed a modest background level of nuclear fluorescence that was greater in the more differentiated FTC 236 cells that in the anaplastic SW-1736 cells. In contrast, FR901228-treated FTC 236 and SW-1736 cells showed intense nuclear fluorescence, reflecting a marked increase in histone acetylation relative to the control cells. Thus, treatment with 1 ng/ml FR901228 markedly increases histone acetylation in both more differentiated and less differentiated thyroid carcinoma cell lines.

### EXAMPLE 3

### Histone Deacetylase Inhibition Activates Thyroid-Specific Promoters

Thyroglobulin (TG) is a thyroid hormone-binding protein produced by normal, fully differentiated thyroid cells, but not by other cell types. Expression of TG is largely regulated at the transcriptional level, by activation of the thyroid-specific TG enhancer-promoter element.

In thyroid carcinoma, thyroid-specific gene expression may be impaired or lost. This loss of thyroid-specific gene expression may be an important factor in maintaining the cancerous phenotype. Agents that promote thyroid-specific gene expression may promote differentiation of thyroid carcinoma cells, thereby reducing the biologically aggressive behavior of these tumors.

To demonstrate the effect of histone deacetylase inhibition on thyroid-specific gene expression, the effect of FR901228 on thyroglobulin promoter activity was shown. This demonstrated that 1 ng/ml FR901228 markedly increased TG promoter-enhancer activity in thyroid carcinoma cells.

Methods and Materials

FTC 236 and SW-1736 cells were transiently transfected with a reporter plasmid encoding luciferase operably linked to a TG promoter-enhancer element. Cells transfected with this reporter plasmid will express luciferase upon activation of the TG promoter enhancer. Luciferase activity in cell lysates accurately reflects TG promoter enhancer activity in the cell.

As a positive control, FTC 236 and SW-1736 cells were transfected with a reporter plasmid encoding luciferase operably linked to the thymidine kinase (TK) promoter enhancer. The TK promoter enhancer is a constitutively active promoter element, meaning that it is not thyroid-specific and is fully active in all cell types.

For transfection, FTC 236 and SW-1736 cells were exposed to a transfection mixture of 0.5 micrograms of plasmid DNA, 4.5 microliters TransFast transfection reagent (Promega, Madison Wis.) and 200 microliters of RPMI medium. Plasmid DNA was either a TG-luciferase (TG-luc) construct or a TK-luciferase (TK-luc) construct. All transfections were performed in triplicate.

After incubating cells for one hour with the transfection mixture, cells were cultured in the presence or absence of 1 ng/ml FR901228 for two days. Cells were then harvested and lysed, and an extract of cellular protein was obtained. Total protein concentration in the extract was determined using the Bio-Rad protein assay system (Bio-Rad, Richmond Calif.). Luciferase activity was determined using the Luciferase Assay System (Promega, Madison Wis.), and was normalized to total protein concentration.

Results

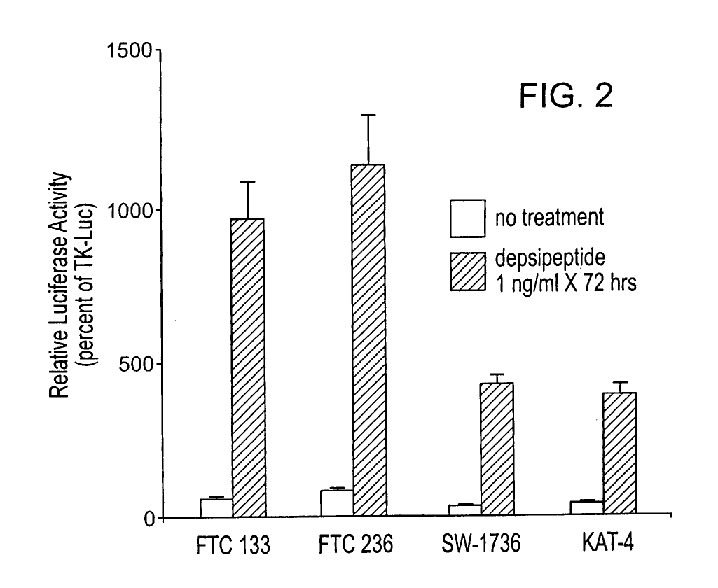

Results are presented in FIG. 2. Normalized luciferase activity in TK luc-transfected cells was assigned a value of 100%, and normalized luciferase activity in TG luc-transfected cells was expressed relative to this as relative luciferase units (RLU).

In the absence of HDI treatment, normalized luciferase activity in TG luc-transfected cells was less than that observed in TK luc-transfected cells. In the relatively well differentiated FTC 133 and FTC 236 cells, RLU in TG luc-transfected cells was about 50-80%. In the less differentiated SW-1736 and KAT-4 cells, TG luc transfection resulted in about 30% RLU. Thus, baseline TG promoter enhancer activity is greater in the more differentiated thyroid carcinoma cells that in the less differentiated anaplastic cells.

Treatment with 1 ng/ml FR901228 had no effect on luciferase activity in TK luc-transfected cells. However, FR901228 treatment markedly increased RLU in TG-luc transfected cells. For example, RLU in FTC 133 and FTC 236 cells was about 1000%, representing a greater than tenfold enhancement in luciferase activity over that observed in untreated cells. In the less differentiated SW-1736 and KAT-4 cells, RLU was 400-500%, again representing a greater than ten-fold enhancement over RLU observed in untreated cells.

These results demonstrate that HDI treatment markedly enhances the activity of the thyroid-specific TG promoter enhancer in thyroid carcinoma cell types.

### EXAMPLE 4

### Histone Deacetylase Inhibition Increases Expression of Thyroid-Specific Genes

Since HDI enhancement of TG promoter activity is physiologically relevant, HDI treatment also increases expression of TG promoter-regulated genes. In this example, it is demonstrated that FR901228 treatment markedly increases expression of two TG promoter-regulated genes.

Materials and Methods

RT PCR and northern blot analysis were used to demonstrate transcriptional regulation of thyroglobulin and Na iodide symporter (Na⁺/I⁻ symporter or NIS) expression in FR901228-treated and untreated thyroid carcinoma cells. RT PCR and Northern blot analysis are described in detail in a number of standard molecular biology reference works, for example Ausubel et al., Short Protocols in Molecular Biology, John Wiley & Sons, 1998.

Total RNA was extracted from FR901228-treated and untreated thyroid carcinoma cells using RNA STAT-60 (Tel-Test, Inc.), at 24 hours, 48 hours, and 72 hours following addition of FR901228-containing or control solution to the cell cultures. RNA was also extracted from normal thyroid cells for purposes of comparison. The extracted RNA was used without further modification for northern blots. For RT PCR, the extracted RNA was reverse transcribed into cDNA using standard techniques.

Oligonucleotide primers used for PCR analysis of human thyroglobulin expression were:

| | | |
|---|---|---|
| TG 5′ (sense) | GAA ATC GTC GTC TTC TCC AC | (SEQ ID NO:1) |
| TG 3′ (antisense) | TGA CGG TGA AGG AGC CCT GAA G | (SEQ ID NO:2) |

Using these primers, the presence of human thyroglobulin cDNA template is reflected by a 219 bp PCR product.

Oligonucleotide primers used for PCR analysis of human Na⁺/I⁻ symporter expression were:

| | | |
|---|---|---|
| NIS (1) 5′ (sense) | CTG CCC CAG ACC AGT ACA TGC C | (SEQ ID NO:3) |
| NIS (1) 3′ (antisense) | TGA CGG TGA AGG AGC CCT GAA G | (SEQ ID NO:4) |

Using these primers, the presence of human Na⁺/I⁻ symporter cDNA template is reflected by a 303 bp PCR product.

Results

In untreated ATC cells, no TG or NIS expression could be detected. Thus, these genes are not expressed in untreated anaplastic thyroid carcinoma cells. In untreated FTC cells, a very faint band could be detected by RT PCR, but not by Northern blot. This indicates very low-level expression of both TG and Na⁺/I⁻ symporter in more differentiated thyroid carcinoma cells.

In FTC cells, increased TG and Na⁺/I⁻ symporter expression was detected by both RT PCR and northern blot analysis within 24 hours following the addition of FR901228, with further increases observed after 72 hours. In ATC cells, increased expression was first observed 48 hours after FR901228, with further increases observed after 72 hours of FR901228 exposure. Control treated cells did not show increased expression of TG or Na⁺/I⁻ symporter.

These experiments provide evidence that HDI treatment markedly enhances expression of thyroid-specific genes in thyroid carcinoma cells.

### EXAMPLE 5

### Inhibition of Histone Deacetylase Increases Expression of the Na-I Symporter

Increased transcription of a gene does not necessarily result in increased levels of functional protein in the cell. Numerous other factors, such as mRNA and protein stability, affect protein concentration. This example demonstrates (1) that a functional Na⁺/I⁻ symporter was expressed in thyroid carcinoma cells, and (2) that Na⁺/I⁻ symporter expression could be increased by HDI treatment.

These experiments revealed that thyroid carcinoma cells contain low levels of functional Na⁺/I⁻ symporter, but these low levels could be markedly increased by HDI treatment.

Methods and Materials

To evaluate the function of the Na⁺/I⁻ symporter, iodine accumulation studies were performed. FTC 133, FTC 236 SW-1736 and KAT-4 cells were treated with 1 ng/ml FR901228 for two or three days, or left untreated. Cells were then incubated in 0.5 ml of Hanks' Balanced Salt Solution (HBSS; Life Technologies, Inc., Eggenstein, Germany) containing approximately 2 μCi carrier-free Na¹²⁵I (DuPont NEN, Boston, Mass.) and 30 μM NaI] ¹²⁵I for 10 minutes. For perchlorate studies NaCIO₄ was added as a 100X solution in HBSS, to a final concentration of 30 and 100 μM, immediately after the addition of radiolabeled iodine. Excess radiolabeled iodine was then removed, and the amount of ¹²⁵I accumulated in the cells was determined by gamma counting.

Results

In the absence of FR901228 treatment, iodine accumulation in FTC cells was higher than that in ATC cells. This result is consistent with the higher expression of Na⁺/I⁻ symporter observed in RT PCR studies (Example 4), and is also consistent with the more differentiated state of FTC cells relative to ATC cells.

Marked increases in iodine accumulation were observed in the four cell lines at two and three days following the addition of FR901228. Iodine accumulation was inhibited in a dose-dependent manner by sodium perchlorate, indicating that the iodine accumulation was a result of Na⁺/I⁻ symporter activity. Thus, in both FTC and ATC thyroid carcinoma cells, histone deacetylase inhibition induced transcription of Na⁺/I⁻ symporter (Example 4), and increased expression of functional Na⁺/I⁻ symporter.

### EXAMPLE 5

### Histone Deacetylase Inhibition in Treatment of Thyroid Carcinoma

The disclosures contained herein enable a novel approach to treatment of thyroid carcinoma in human and animal subjects. Administration of histone deacetylase inhibitors has been shown herein to have salutary effects that may be exploited in thyroid cancer therapy. For example, HDI administration induces thyroid carcinoma cells to differentiate, thereby decreasing biologically aggressive behaviors such as rapid growth and tendency to metastasize. In addition, HDI administration increases levels of functional Na⁺/I⁻ symporter and TG in thyroid carcinoma cells. Cells expressing functional Na⁺/I⁻ symporter accumulate more iodine, and are therefore more susceptible to radioactive iodine therapy. Increased expression of TG also enhances intercellular iodine accumulation in thyroid carcinoma cells.

Protocol For HDI Inhibitor Therapy Combined With ¹³¹I Radiotherapy

A subject with a thyroid carcinoma is administered a therapeutically effective amount of a histone deacetylase inhibitor. If desired, therapeutic efficacy of HDI therapy is monitored by radionucleotide scans after administration of a “tracer,” for example a diagnostic dose of ¹³¹I (for example as described in McDougall et al., Nucl Med Commun 18: 505-510, 1997). Other suitable tracers include ¹²³I and ^{99m}Tc-labeled pertechnitate. Such radionuclide scans can be performed before, during and after HDI inhibitor therapy, to follow and quantitate increases in tracer accumulation after administration of tracer. HDI inhibitor therapy increases tracer accumulation by about twofold, about fivefold, about tenfold, or greater than tenfold.

In one embodiment, the subject has not received prior therapy. In another embodiment, the subject has previously undergone a thyroidectomy and/or radioactive iodine therapy for thyroid carcinoma. In some instances the subject is a subject who has or is suspected of having residual and/or metastatic thyroid carcinoma at one or more sites in the body, and the method is a method of treating the residual and/or metastatic thyroid carcinoma.

After or concurrent with treatment with a therapeutically effective amount of a histone deacetylase inhibitor, radioactive iodine therapy is administered. Radioiodine treatment may be accompanied by discontinuation of thyroid hormone replacement, thereby inducing clinical hypothyroidism. The hypothyroid state triggers pituitary secretion of thyroid stimulating hormone (TSH), which will be additive or synergistic with HDI therapy in increasing Na⁺/I⁻ symporter activity and TG expression, thereby further inducing increased uptake and intercellular accumulation of ¹³¹I . As one alternative, TSH may also be administered exogenously, for example as described in Meier et al., J Clin Endocrinol Metab 78: 188, 1994. Exogenous TSH is also additive or synergistic with HDI therapy in increasing Na⁺/I⁻ symporter activity and TG expression.

Radioactive iodine therapy is often administered as large doses of ¹³¹I (for example, 50 to 500 mCi to a 70 kg human subject). HDI enhancement of intracellular radioactive iodine accumulation enhances the efficacy of radioactive iodine therapy, or enables lower doses to be administered without loss of antitumor effect. For example, if HDI therapy increases iodine accumulation in thyroid carcinoma cells by fivefold, it is possible to reduce the administered dose of ¹³¹I , for example to 1 to 50 mCi to a 70 kg human subject. Size of dose may be adjusted for weight, body surface area and/or species.

In addition to radioactive iodine therapy, HDI therapy can be combined with any other therapy. For example, a subject receiving HDI therapy may receive external irradiation or anticancer chemotherapy at about the same time as the HDI therapy, or before or after HDI therapy. HDI therapy can be additive or synergistic with these other therapeutic modalities.

Following administration of radioactive iodine, the subject is hypothyroid, due to radiation-induced death of thyroid cells and the fact that most subjects have also undergone thyroidectomy. Therefore, thyroid hormone replacement therapy is initiated or restarted.

This or a similar protocol may be repeated at intervals (for example about every 2-24 months, or about every 6-12 months), particularly if residual thyroid cancer is present.

### EXAMPLE 6

### HDI Inhibition Using Antisense Oligonucleotides

This example employs antisense compounds, particularly oligonucleotides, for use in modulating the function of nucleic acid molecules encoding histone deacetylase, ultimately reducing the amount of histone deacetylase produced (see WO 0071703A2, which is herein incorporated by reference). This is accomplished by providing oligonucleotides which specifically hybridize with nucleic acids, preferably mRNA, encoding a histone deacetylase isoform.

This relationship between an antisense compound such as an oligonucleotide and its complementary nucleic acid target, to which it hybridizes, is commonly referred to as “antisense.” “Targeting” an oligonucleotide to a chosen nucleic acid target usually begins with identifying a nucleic acid sequence whose function is to be modulated. Histone deacetylase mRNA is presently the preferred target. Histone deacetylase mRNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Oligonucleotides are chosen which are sufficiently complementary to the target, i.e., hybridize sufficiently well and with sufficient specificity, to function as a therapeutically effective oligonucleotide. “Hybridization,” in the context of this disclosure, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them.

“Specifically hybridizable” and “complementary” are terms which are used to indicate a sufficient degree of complementarity such that stable and specific binding occurs between the DNA or RNA target and the oligonucleotide.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

To design an antisense oligonucleotide, the mRNA sequence from the desired molecule, such as histone deacetylase, is examined. Regions of the sequence containing multiple repeats, such as TTTTTTTT, are not as desirable because they will lack specificity. Several different regions can be chosen. Of those, oligonucleotides are selected by the following characteristics: ones having the best conformation in solution; ones optimized for hybridization characteristics; and one having less potential to form secondary structures. Antisense molecules having a propensity to generate secondary structures are less desirable.

This modulation can be measured in ways which are routine in the art, for example by Northern blot assay of histone deacetylase mRNA expression, or reverse transcriptase PCR, as taught in the examples of the instant application or by Western blot or ELISA assay of histone deacetylase protein expression, or by an immunoprecipitation assay of histone deacetylase protein expression.

Specific examples of antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and borano-phosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248.

In view of the many possible embodiments to which the principles of the invention may be applied, it should be recognized that the illustrated embodiments are examples of the invention, and should not be taken as a limitation on the scope of the invention. Rather, the scope of the invention is defined by the following claims. We therefore claim as our invention all that comes within the scope and spirit of these claims.

We claim:

1. A method of enhancing expression of a thyroid specific gene in a thyroid cell, comprising introducing into a thyroid cell an effective amount of an agent that inhibits histone deacetylase, thereby enhancing expression of the thyroid specific gene.

2. The method of claim 1, wherein the thyroid specific gene is Na⁺/I⁻ symporter.

3. The method of claim 1, wherein the thyroid specific gene is thyroglobulin.

4. The method of claim 1, wherein enhancing expression of the thyroid specific gene increases ability of the thyroid cell to take up and/or concentrate iodide or iodine.

5. The method of claim 1, wherein the agent comprises FR901228 (depsipeptide), trichostatin A, trapoxin A, trapoxin B, HC-toxin, chlamydocin, Cly-2, WF-3161, Tan-1746, apicidin, analogs of apicidin, benzamide, derivatives of benzamide, hydroxyamic acid derivatives, azelaic bishydroxyamic acid, butyric acid and salts thereof, actetate salts, suberoylanilide hydroxyamide acid, suberic bishydroxyamic acid, m-carboxy-cinnamic acid bishyrdoxyamic acid, oxamflatin, depudecin, or MS-27-275.

6. The method of claim 1, wherein the agent comprises FR901228 (depsipeptide).

7. The method of claim 1, wherein the agent comprises an oligonucleotide that inhibits expression or function of histone deacetylase.

8. The method of claim 1, wherein the agent comprises a dominant negative fragment or variant of histone deacetylase.

9. The method of claim 1, wherein enhancing the expression of the thyroid specific gene increases iodine or iodide uptake by the cell.

10. The method of claim 9, wherein the thyroid cell is a thyroid cancer cell.

11. The method of claim 9, wherein the thyroid cell is in vitro.

12. A method of treating a thyroid cancer in a subject, comprising administering to the subject a therapeutically effective amount of an agent that inhibits histone deacetylase, thereby treating the thyroid cancer in the subject.

13. The method of claim 12, further comprising administering a therapeutically effective amount of a radioactive iodine to the subject, wherein the administration of the agent that inhibits histone deaceylase increases uptake and/or concentration of the radioactive iodine in a neoplastic cell in the thyroid cancer.

14. The method of claim 13, wherein the radioactive iodine is ¹³¹I.

15. The method of claim 12, wherein about 5 mCi to about 500 mCi of radioactive iodine is administered to the subject.

16. The method of claim 12, wherein about 30 mCi to about 300 mCi of radioacitve iodine is administered to the subject.

17. The method of claim 12, wherein the thyroid cancer is a papillary thyroid carcinoma or histologic variant thereof, a follicular thyroid carcinoma or histologic variant thereof, an insular thyroid carcinoma or histologic variant thereof, or an anaplastic thyroid carcinoma or histologic variant thereof.

18. The method of claim 12, wherein the subject has undergone or will undergo thyroidectomy.

19. The method of claim 12, wherein the thyroid cancer is a residual thyroid carcinoma that remains after a thyroidectomy.

20. The method of claim 12, wherein the thyroid cancer is a metastatic thyroid carcinoma.

21. The method of claim 12, wherein the radioactive iodine is administered to the subject in a plurality of doses.

22. The method of claim 11, further comprising administering to the subject a therapeutically effective amount of a chemotherapeutic agent.

23. A method of detecting a neoplastic thyroid cell in a subject, comprising:
administering to the subject an effective amount of an agent that inhibits histone deacetylase;
administering to the subject a detectable agent that is taken up by the neoplastic thyroid cell, wherein uptake or concentration of the detectable agent in the neoplastic thyroid cell is increased by the agent that inhibits histone deacetylase; and
detecting the detectable agent in the neoplastic thyroid cell.

24. The method of claim 21, wherein the neoplastic cell is a cell in a thyroid carcinoma.

25. The method of claim 23, wherein the thyroid carcinoma is a papillary thyroid carcinoma, a follicular thyroid carcinoma, an insular thyroid carcinoma, or an anaplastic thyroid carcinoma.

26. The method of claim 23, wherein the detectable agent comprises an agent that is transported via a Na⁺/I⁻ symporter into the thyroid cell.

25. The method of claim 23, wherein the detectable agent is a radioactive iodine molecule.

26. The method of claim 23, wherein the detectable agent is ¹²³I, ¹²⁵I, ¹³¹I , radiolabeled perchlorate, or radiolabeled pertechnitate.

27. The method of claim 23, wherein the neoplastic cell is a residual neoplastic cell in a subject that has received therapy for a thyroid carcinoma.

28. The method of claim 27, wherein the therapy for thyroid carcinoma is thyroidectomy, ¹³¹I therapy, external radiation, or administration of an anticancer chemotherapeutic agent.

29. A method of increasing the uptake of iodine in the thyroid of a subject comprising administering a therapeutically effective amount of an agent that inhibits a histone deacetylase inhibitor, thereby increasing iodine or iodide uptake.

30. The method of claim 25, wherein the iodine is radioactive.

31. The method of claim 26, wherein the iodine is radioactive iodine is ¹³¹I.

32. Use of an agent that inhibits a histone deacetylase for the treatment of thyroid cancer.

33. Use of an agent that inhibits a histone deacetylase to increase the uptake of iodine or iodide in the thyroid.

34. The use of claim 32 or 33, wherein the agent comprises FR901228, trichostatin A, trapoxin A, trapoxin B, HC-toxin, chlamydocin, Cly-2, WF-3161, Tan-1746, apicidin, analogs of apicidin, benzamide, derivatives of benzamide, hydroxyamic acid derivatives, azelaic bishydroxyamic acid, butyric acid and salts thereof, actetate salts, suberoylanilide hydroxyamide acid, suberic bishydroxyamic acid, m-carboxy-cinnamic acid bishyrdoxyamic acid, oxamflatin, depudecin, or MS-27-275.


SEQUENCE LISTING (4 sequences)

SEQ ID NO: 1 - DNA, 20 bp, Artificial Sequence
  Oligonucleotide primer
gaaatcgtcgtcttctccac

SEQ ID NO: 2 - DNA, 22 bp, Artificial Sequence
  Oligonucleotide primer
tgacggtgaaggagccctgaag

SEQ ID NO: 3 - DNA, 22 bp, Artificial Sequence
  Oligonucleotide primer
ctgccccagaccagtacatgcc

SEQ ID NO: 4 - DNA, 22 bp, Artificial Sequence
  Oligonucleotide primer
tgacggtgaaggagccctgaag